(19) United States
(12) Patent Application Publication
(10) Pub. No.: US 2024/0136063 A1
(43) Pub. Date: Apr. 25, 2024

(54) EVIDENCE-BASED OUT-OF-DISTRIBUTION DETECTION ON MULTI-LABEL GRAPHS

(71) Applicant: NEC Laboratories America, Inc., Princeton, NJ (US)

(72) Inventors: Xujiang Zhao, Hillsborough, NJ (US); Haifeng Chen, West Windsor, NJ (US)

(21) Appl. No.: 18/481,383

(22) Filed: Oct. 5, 2023

Related U.S. Application Data
(60) Provisional application No. 63/413,695, filed on Oct. 6, 2022.

(51) Int. Cl. G16H 50/20; G16H 50/70

(52) U.S. Cl. CPC G16H 50/20; G16H 50/70

(57) ABSTRACT

Systems and methods for out-of-distribution detection of nodes in a graph includes collecting evidence to quantify predictive uncertainty of diverse labels of nodes in a graph of nodes and edges using positive evidence from labels of training nodes of a multi-label evidential graph neural network. Multi-label opinions are generated including belief and disbelief for the diverse labels. The opinions are combined into a joint belief by employing a comultiplication operation of binomial opinions. The joint belief is classified to detect out-of-distribution nodes of the graph. A corrective action is performed responsive to a detection of an out-of-distribution node. The systems and methods can employ evidential deep learning.

### RELATED APPLICATION INFORMATION

This application claims priority to U.S. Provisional Application No. 63/413,695, filed on Oct. 6, 2022, incorporated herein by reference in its entirety.

### BACKGROUND

### Technical Field

The present invention relates to graph structured data systems and methods and more particularly to graph structured networks that address nodes that include out of distribution nodes.

### Description of the Related Art

Many real-world application scenarios can be represented by graph structured data, ranging from natural networks to social networks. In graph scenarios, there are usually only a subset of nodes that are labeled. Multi-label properties of nodes cannot be avoided. For example, in social networks, one user may have more than one interest. In a Protein-Protein-Interaction (PPI) network, one protein can perform multiple functions. Since unknown labels are unavoidable, some of the unlabeled nodes may be out-of-distribution (OOD) and need to be discovered.

### SUMMARY

According to an aspect of the present invention, a method for out-of-distribution detection of nodes in a graph includes collecting evidence to quantify predictive uncertainty of diverse labels of nodes in a graph of nodes and edges using positive evidence from labels of training nodes of a multi-label evidential graph neural network. Multi-label opinions are generated including belief and disbelief for the diverse labels. The opinions are combined into a joint belief by employing a comultiplication operation of binomial opinions. The joint belief is classified to detect out-of-distribution nodes of the graph. A corrective action is performed responsive to a detection of an out-of-distribution node.

According to another aspect of the present invention, a system for out-of-distribution detection of nodes in a graph includes a hardware processor and a memory that stores a computer program which, when executed by the hardware processor, causes the hardware processor to collect evidence to quantify predictive uncertainty of diverse labels of nodes in a graph of nodes and edges using positive evidence from labels of training nodes of a multi-label evidential graph neural network; generate multi-label opinions including belief and disbelief for the diverse labels; combine the opinions into a joint belief by employing a comultiplication operation of binomial opinions; and classify the joint belief to detect out-of-distribution nodes of the graph.

According to another aspect of the present invention, a computer program product for out-of-distribution detection of nodes in a graph, the computer program product comprising a non-transitory computer readable storage medium having program instructions embodied therewith, the program instructions executable by a computer to cause the computer to perform a method including collecting evidence to quantify predictive uncertainty of diverse labels of nodes in a graph of nodes and edges using positive evidence from labels of training nodes of a multi-label evidential graph neural network; generating multi-label opinions including belief and disbelief for the diverse labels; combining the opinions into a joint belief by employing a comultiplication operation of binomial opinions; classifying the joint belief to detect out-of-distribution nodes of the graph; and performing a corrective action responsive to a detection of an out-of-distribution node.

These and other features and advantages will become apparent from the following detailed description of illustrative embodiments thereof, which is to be read in connection with the accompanying drawings.

### BRIEF DESCRIPTION OF DRAWINGS

The disclosure will provide details in the following description of preferred embodiments with reference to the following figures wherein:

FIG. 1 is a block/flow diagram illustrating a high-level system/method for evidence-based out-of-distribution detection on multi-label graphs, in accordance with an embodiment of the present invention;

FIG. 2 is a block/flow diagram illustrating a system/method for an out-of-distribution detection system on graph-structured data, in accordance with an embodiment of the present invention;

FIG. 3 is a flow diagram illustrating a method for detecting out-of-distribution nodes in graphs, in accordance with an embodiment of the present invention;

FIG. 4 is an illustrative example of a Protein-Protein Interaction (PPI) network employing a multi-label evidential graph neural network to improve the performance of node-level multi-label out-of-distribution detection, in accordance with an embodiment of the present invention;

FIG. 5 is a block diagram showing a medical system that employs a multi-label evidential graph neural network to improve the performance of node-level multi-label out-of-distribution detection, in accordance with an embodiment of the present invention;

FIG. 6 is a block diagram showing an exemplary processing system employed in accordance with an embodiment of the present invention;

FIG. 7 is a generalized illustrative diagram of a neural network, in accordance with an embodiment of the present invention; and

FIG. 8 is a flow diagram illustrating a method for detecting out-of-distribution nodes in graphs, in accordance with an embodiment of the present invention.

### DETAILED DESCRIPTION OF PREFERRED EMBODIMENTS

Embodiments in accordance with the present invention address Out-of-Distribution (OOD) detection on graph-structured data. OOD is an issue in various areas of research and applications including social network recommendations, protein function detection, medication classification, medical monitoring and other graph-structured data applications. The inevitable inherent multi-label properties of nodes provides more challenges for multi-label OOD detection than multi-class settings. Existing OOD detection methods on graphs are not applicable for multi-label settings. Other semi-supervised node classification methods lack the ability to differentiate OOD nodes from in-distribution (ID) nodes. Multi-class classification assigns each data sample one and only one label from more than two classes. Multi-label classification can be used to assign zero or more labels to each data sample.

Out-of-distribution detection on multi-label graphs, in accordance with the present embodiments, can incorporate Evidential Deep Learning (EDL) to provide a novel Evidence-Based OOD detection method for node-level classification on multi-label graphs. The evidence for multiple labels is predicted by Multi-Label Evidential Graph Neural Networks (ML-EGNNs) with beta loss. A Joint Belief is designed for multi-label opinions fusion by a comultiplication operator. Additionally, a Kernel-based Node Positive Evidence Estimation (KNPE) method can be introduced to reduce errors in quantifying positive evidence. Experimental results prove both the effectiveness and efficiency of our model on multi-label OOD Detection. Also, the present methods can maintain an ideal close-set classification performance when compared with baselines on real-world multi-label networks.

Learning methods for multi-label node classification on graphs to predict user interests in social networks, classify medical conditions, identify functions of proteins in PPI networks, etc. are capable of differentiating OOD nodes from in-distribution (ID) nodes. By effectively distinguishing OOD nodes, users with potential interests, for example, can be identified for better recommendations or unknown functions of proteins can be discovered for pharmaceutical research. In a particularly useful embodiment, medical information can be employed in a graphical setting where each node can include a patient or user or characteristics of a patient or user. Multiple labels for each patient may need to be evaluated to ensure all of the patient's medical conditions are properly classified.

Multi-Label Out-of-Distribution Detection can be employed for data mining and network analysis. The OOD samples can be connected with low belief and lack of classification evidence from Subjective Logic (SL). Multi-label Out-of-Distribution on graphs can be trained on: (1) how to learn evidence or belief for each possibility based on structural information and node features; (2) how to combine information from different labels and comprehensively decide whether a node is out-of-distribution; (3) how to maintain ideal close-set multi-label classification results while effectively performing OOD detection.

In one embodiment, an evidential OOD detection method for node-level classification tasks on multi-label graphs is provided. Evidential Deep Learning (EDL) is leveraged in which the learned evidence is informative to quantify the predictive uncertainty of diverse labels so that unknown labels would incur high uncertainty. Beta distributions can be introduced to make Multi-Label Evidential Graph Neural Networks (ML-EGNNs) feasible. Joint Belief is formulated for multilabel samples by a comultiplication operator of binomial opinions, which combines argument opinions from multiple labels. The separate belief of classes obtained by evidential neural networks are employed as a basis for close-set classification, which is both effective and efficient.

A Kernel-based Node Positive Evidence Estimation (KNPE) method uses structural information and prior positive evidence collected from the given labels of training nodes, to optimize a neural network model and to help detect multi-label OOD nodes. A method for node-level OOD detection uses a multi-label evidential neural network, in which OOD conditions can be directly inferred from evidence prediction, instead of relying on time-consuming dropout or ensemble techniques.

OOD detection on multi-label graphs using evidential methods for the multi-label node-level detection are provided. Evidential neural networks are utilized with beta loss to predict the belief for multiple labels. Joint Belief is defined for multi-label opinions fusion. Further, a Kernel-based Node Positive Evidence Estimation (KNPE) method is provided to reduce errors in quantifying positive evidence.

Experimental results prove both the effectiveness and efficiency of models, in accordance with the present embodiments, on multi-label OOD detection, which is able to maintain an ideal close-set classification level when compared with baselines on real-world multi-label networks.

Embodiments described herein may be entirely hardware, entirely software or including both hardware and software elements. In a preferred embodiment, the present invention is implemented in software, which includes but is not limited to firmware, resident software, microcode, etc.

Embodiments may include a computer program product accessible from a computer-usable or computer-readable medium providing program code for use by or in connection with a computer or any instruction execution system. A computer-usable or computer readable medium may include any apparatus that stores, communicates, propagates, or transports the program for use by or in connection with the instruction execution system, apparatus, or device. The medium can be magnetic, optical, electronic, electromagnetic, infrared, or semiconductor system (or apparatus or device) or a propagation medium. The medium may include a computer-readable storage medium such as a semiconductor or solid state memory, magnetic tape, a removable computer diskette, a random access memory (RAM), a read-only memory (ROM), a rigid magnetic disk and an optical disk, etc.

Each computer program may be tangibly stored in a machine-readable storage media or device (e.g., program memory or magnetic disk) readable by a general or special purpose programmable computer, for configuring and controlling operation of a computer when the storage media or device is read by the computer to perform the procedures described herein. The inventive system may also be considered to be embodied in a computer-readable storage medium, configured with a computer program, where the storage medium so configured causes a computer to operate in a specific and predefined manner to perform the functions described herein.

A data processing system suitable for storing and/or executing program code may include at least one processor coupled directly or indirectly to memory elements through a system bus. The memory elements can include local memory employed during actual execution of the program code, bulk storage, and cache memories which provide temporary storage of at least some program code to reduce the number of times code is retrieved from bulk storage during execution. Input/output or I/O devices (including but not limited to keyboards, displays, pointing devices, etc.) may be coupled to the system either directly or through intervening I/O controllers.

Network adapters may also be coupled to the system to enable the data processing system to become coupled to other data processing systems or remote printers or storage devices through intervening private or public networks. Modems, cable modem and Ethernet cards are just a few of the currently available types of network adapters.

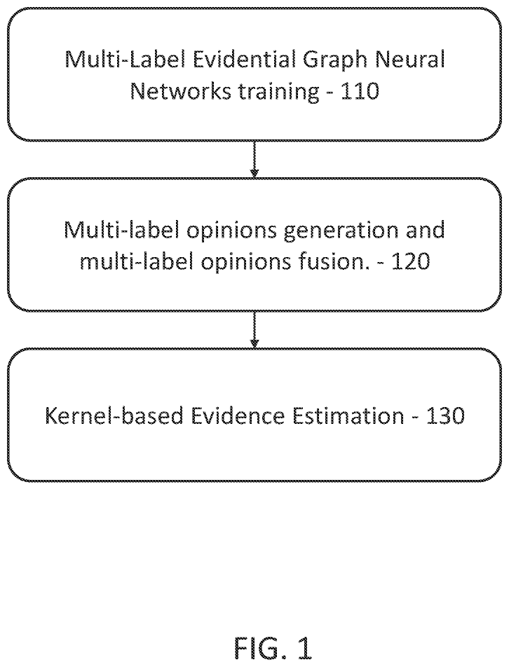

Referring now in detail to the figures in which like numerals represent the same or similar elements and initially to FIG. 1, a high-level system/method for evidence-based out-of-distribution detection on multi-label graphs is shown in accordance with embodiments of the present invention. Multi-label out-of-distribution detection is performed using a multi-label evidential neural network method. Given multi-label graph data, a goal is to detect the out-of-distribution nodes. This is done by minimizing an area under a precision-recall curve (AUPR) for out-of-distribution detection to make a prediction more accurate.

To address node level out-of-distribution detection on multi-label graph data, one embodiment provides a new Multi-Label Evidential Graph Neural Networks (ML-EGNN) framework 100 that utilizes evidential neural networks with beta loss to predict a belief for multiple labels. In block 110, the framework leverages evidential deep learning in which learned evidence is informative to quantify a predictive uncertainty of diverse labels so that unknown labels would incur high uncertainty and thus provide a basis for differentiating the diverse labels. Beta distributions are also introduced to make the model feasible. In block 120, the framework provides joint belief for multi-label samples by a comultiplication operator of binomial opinions, which combines argument opinions from multiple labels.

In block 130, kernel-based node positive evidence estimation is provided and uses structural information, and prior positive evidence that was collected from the given labels of training nodes, to help detect multi-label out-of-distribution nodes. Experimental results show the effectiveness and efficiency of the model on multi-label OOD detection. The framework can maintain an ideal close-set classification level when compared with baselines on real-world multi-label networks.

Block 110 provides multi-label node evidence estimation. In this step, a Multi-Label Evidential Graph Neural Network (ML-EGNN) is designed and built by stacking graph convolutional layers and two fully connected layers (FCs) and rectified linear unit (ReLU) layers.

Neurons in a ML-EGNN can include a respective activation function. These activation functions represent an operation that is performed on an input of a neuron, and that help to generate the output of the neuron. Here, the activation function can include ReLU but other appropriate activation functions may be adapted for use. ReLU provides an output that is zero when the input is negative, and reproduces the input when the input is positive. The ReLU function notably is not differentiable at zero—to account for this during training, the undefined derivative at zero may be replaced with a value of zero or one.

The node evidence estimation output from the graph convolutional layers, FCs and ReLU layers is taken as the positive and negative evidence vectors for Beta distribution, respectively. Given sample i, let fₚₒₛ(X, A|θ) and f_{neg}(X, A|θ) represent the positive and negative evidence vectors predicted by Evidential Graph Neural Networks (EGNNs), where X is the input node features, A is the adjacency matrix, and θ represents the network parameters. Then, the parameters of the Beta distribution for node i and label k are:

αᵢₖ=fₚₒₛ(X, A|θ)+1,

βᵢₖ=f_{neg}(X, A|θ)+1.

With N training samples and K different classes, a multi-label evidential neural network is trained by minimizing the Beta Loss:

$$\begin{matrix}{ℒ}_{Beta} = {\sum }_{i = 1}^{N} {\sum }_{k = 1}^{K} \int [ B C E ( {y}_{ik} , {p}_{i k} ) ] B ( {α}_{i k} , {β}_{ik} ) {dp}_{ik} \\ = {\sum }_{j = 1}^{N} {\sum }_{i = 1}^{K} [ {y}_{ij} ( ψ ( {α}_{ij} + {β}_{ij} ) - ψ ( {α}_{ij} ) ) + ( 1 - {y}_{ij} ) ( ψ ( {α}_{ij} + {β}_{ij} ) - ψ ( {β}_{ij} ) ) ] .\end{matrix}$$

where BCE denotes the Binary Cross Entropy Loss. pᵢₖ represents the predicted probability of sample i belonging to class k by model. yᵢₖrepresents the ground truth for sample i with label k, i.e., yᵢₖ=1 means the training node i belongs to class k, otherwise yᵢₖ=0. And ψ(⋅) denotes the Digamma function. Besides, as the belief bᵢₖ and disbelief dᵢₖ of label k for sample i, then:

$${b}_{i k} = \frac{{α}_{ik} - 1}{{α}_{i k} + {β}_{ik}} ,$$

$${d}_{i k} = \frac{{β}_{i k} - 1}{{α}_{i k} + {β}_{i k}} .$$

For the following process, these beliefs are regarded as multi-label opinions, to formulate a Joint Belief and quantify OOD samples.

In block 120, multi-label opinion fusion is performed. After obtaining separate beliefs of multiple labels, next these opinions are combined and an integrated opinion is quantified, e.g., Opinions Fusion. Let X={x₁, x₂} and Y={y₁, y₂} be two different domains, and let ωₓ=(bₓ, dₓ,uₓ, aₓ) and ω_{y}=(b_{y}, d_{y},u_{y}, a_{y}) be binomial opinions on X and Y respectively. Then, the joint opinion ω_{x∨y} can be formulated as:

$${b}_{x ∨ y} = {b}_{x} + {b}_{y} - {b}_{x} {b}_{y} ,$$

$${d}_{x ∨ y} = {d}_{x} {d}_{y} + \frac{{a}_{x} ( 1 - {a}_{y} ) {d}_{x} {u}_{y} + ( 1 - {a}_{x} ) {a}_{y} {u}_{x} {d}_{y}}{{a}_{x} + {a}_{y} - {a}_{x} {a}_{y}} ,$$

$${u}_{x ∨ y} = {u}_{x} {u}_{y} + \frac{{a}_{y} {d}_{x} {u}_{y} + {a}_{x} {u}_{x} {d}_{y}}{{a}_{x} + {a}_{y} - {a}_{x} {a}_{y}} ,$$

$${a}_{x ∨ y} = {a}_{x} + {a}_{y} - {a}_{x} {a}_{y} .$$

The Joint Belief of a certain sample i is b_{1∨2∨ . . . ∨K}, can be calculated by the above equation recursively.

In block 130, kernel-based evidence estimation is performed. Kernel-based Evidence Estimation estimates prior Beta distribution parameters for each node based on the labels of a training node and node-level distance. The focus is on the estimation of positive evidence \hat{α}. For each pair of nodes i and j, calculate a node-level distance dᵢⱼ, i.e., the shortest path between nodes i and j. Then, a Gaussian kernel function is used to estimate the positive distribution effect between nodes i and j:

$$g ( {d}_{i j} ) = \frac{1}{σ \sqrt{2 π}} exp ( - \frac{{d}_{i j}^{2}}{2 {σ}^{2}} ) ,$$

where σ is the bandwidth parameter. The contribution of positive evidence estimation for node j from training node i is hᵢⱼ(yᵢ, dᵢⱼ)=[hᵢⱼ¹, hᵢⱼ², . . . , hᵢⱼ^{k}, . . . , hᵢⱼ^{K}], where [yᵢ₁, . . . , yᵢₖ, . . . , y_{iK}]=[0, 1]^{K} represents the in-distribution label vector of training node i, and hᵢⱼ^{k} is obtained by:

$${h}_{ij}^{k} = \{ \begin{matrix}0 & {y}_{i k} = 0 \\ g ( {d}_{ij} ) & {y}_{i k} = 1\end{matrix} .$$

The prior positive evidence êⱼ is estimated as all hᵢⱼ^{k} in a set of training samples. During the training process, Kullback—Leibler (KL) divergence (KL-divergence) is minimized between model predictions of positive evidence and prior positive evidence. KL-divergence (also called relative entropy or I-divergence), denoted, is a statistical distance of how one probability distribution P is different from a reference probability distribution Q. A relative entropy of 0 indicates that the two distributions in question have identical quantities of information. Relative entropy is a non-negative function of two distributions or measures.

A total loss function (e.g., sum of beta loss and weighted positive evidence loss) that can be used to optimize the model can include:

min [symbol] ₜₒₜₐₗ= [symbol] _{Beta}+λ [symbol] _{PE},

[symbol]

_{PE}=Σⱼ₌₁^{N}KL(\hat{α}ⱼ∥αⱼ)

where λ denotes a trade-off parameter.

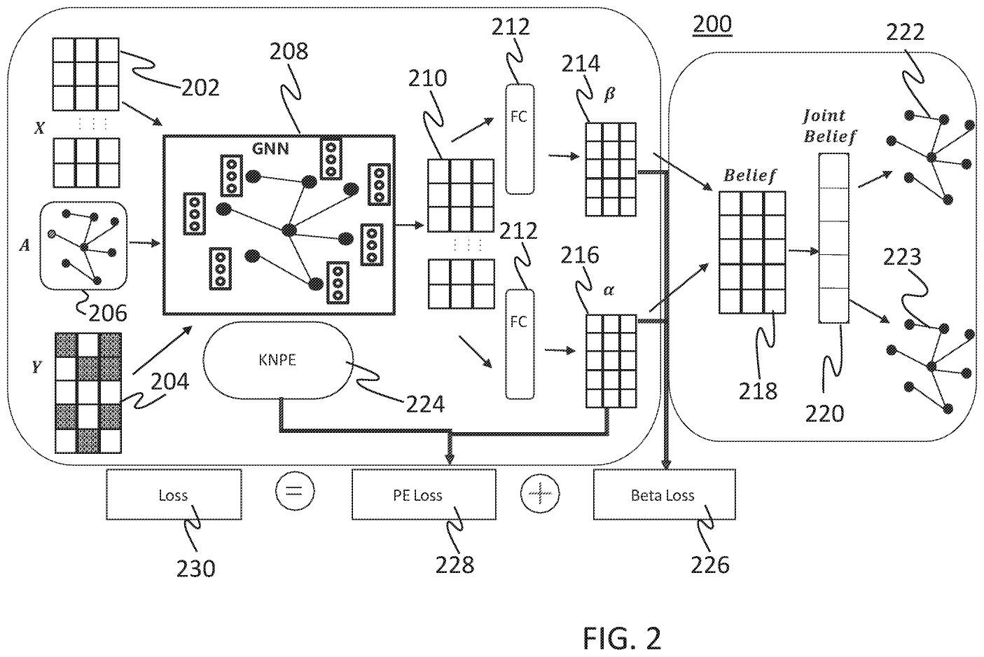

Referring to FIG. 2, a block/flow diagram shows an OOD detection system 200 on graph-structured data. OOD samples can be connected with low belief and lack of classification evidence from Subjective Logic (SL). Subjective logic (SL) is a type of probabilistic logic that explicitly takes epistemic uncertainty and source trust into account. Specifically, epistemic uncertainty measures whether input data exists within the distribution of data already seen. A multinomial opinion of a random variable y is represented by ω=(b, u, a) where a domain is Y={1, . . . , K}, where b indicates belief mass distribution, u indicates uncertainty with a lack of evidence, and a indicates base rate distribution. For a K multiclass setting, a probability mass p=[p₁, p₂, . . . ,p_{K}] is assumed to follow a Dirichlet (Dir (α)) distribution parameterized by a K-dimensional Dirichlet strength vector α={α₁, . . . ,α_{K}}:

$$D i r ( p \vert α ) = \{ \begin{matrix}\frac{1}{B ( α )} {\prod }_{k = 1}^{K} {ρ}_{k}^{{a}_{k} - 1} , & for p ∈ {S}_{K} \\ 0 , & otherwise\end{matrix}$$

where B(α) is a K-dimensional Beta function, S_{K} is a K-dimensional unit simplex. The total strength of the Dirichlet is defined as S=Σₖ₌₁αₖ. Dirichlet distribution is a family of continuous multivariate probability distributions parameterized by a vector of positive reals.

The term evidence indicates how much data supports a particular classification of a sample based on the observations it contains. Let eᵢ={e₁, . . . , e_{K}} be the evidence for K classes. Each entry eₖ≥0 and the Dirichlet strength a are linked according to the evidence theory by the following α=e+aW, where W is the weight of uncertain evidence. With loss of generality, the weight W is set to K and considering the assumption of the subjective opinion that aₖ=1/K, we have the Dirichlet strength αₖ=eₖ+1. The Dirichlet evidence can be mapped to the subjective opinion by setting the following equality's:

$${b}_{k} = \frac{{e}_{k}}{S} and u = \frac{K}{S} .$$

Graph neural networks (GNNs) 208 provide a feasible way to extend deep learning methods into the non-Euclidean domain including graphs and manifolds. The most representative models are, according to the types of aggregators, e.g., Graph Convolutional Network (GCN), Graph Attention Networks (GAT), and GraphSAGE.

It is possible to apply GNNs 208 to various types of training frameworks, including (semi) supervised or unsupervised learning, depending on the learning tasks and label information available. of these, relevant to the present problem is semi-supervised learning for node-level classification. Assuming a network with partial nodes labeled and others unlabeled, GNNs 208 can learn a model that effectively identifies the labels for the unlabeled nodes. In this case, an end-to-end framework can be built by stacking graph convolutional layers 210 followed by fully connected FC layers 214.

Based on Subjective Logic and Belief Theory, the K-dimensional Dirichlet probability distribution function (PDF) is applied for estimating multinomial probability density over a domain of Y={1, . . . , K}. However, it is not feasible for multi-label classification. For Dirichlet distribution, an in-distribution node with multiple labels could be differentiated from other in-distribution samples according to its conflicting evidence, though it shows no sign of lacking evidence. To this end, a Beta distribution is introduced which is able to provide binary evidence for each class.

$$Beta ( p \vert α , β ) = \{ \begin{matrix}\frac{1}{B ( α , β )} {{p}^{α - 1} ( 1 - p )}^{β - 1} , & for p ∈ [ 0 , 1 ] \\ 0 , & otherwise\end{matrix}$$

where the probability mass p∈[0,1] is assumed to follow a Beta distribution parameterised by a 2-dimensional strength vector [α,β]. Belief 218 (B(α,β) is a 2-dimensional Beta function based on α 214 and β 216 the positive and negative evidence vectors, respectively.

Further, a multi-label classification problem Ω can be formalized as a combination of K binomial classifications {ω₁, . . . , ωₖ, . . . , ω_{K}}. Each binomial classification ωₖ holds a binomial opinion: ωₖ=(bₖ, dₖ, uₖ, aₖ) where a domain is Y={0,1}, bₖ indicates positive belief mass distribution, dₖ indicates negative belief mass distribution, uₖ indicates uncertainty with a lack of evidence, and aₖ indicates base rate distribution. The total strength of the Beta is defined as Sₖ=αₖ +βₖ. Then, the Beta evidence can be mapped to a binomial subjective opinion by setting the following equalities:

$${b}_{k} = \frac{{α}_{k} - 1}{{α}_{k} + {β}_{k}} and {d}_{k} = \frac{{β}_{k} - 1}{{α}_{k} + {β}_{k}} ,$$

$${u}_{k} = \frac{2}{{S}_{k}} = \frac{2}{{a}_{k} + {β}_{k}} .$$

Compared with classical Neural Networks, Evidential Neural Networks (ENNs) do not have a softmax layer, but use an activation layer (e.g., ReLU) to make sure that the output is non-negative. Multi-Label Evidential Graph Neural Networks (ML-EGNNs) are built by stacking graph convolutional layers in GNN 208 and two fully connected layers (FCs) 212 with ReLU layers, which are taken as the positive and negative evidence vectors (α 214 and β 216, respectively) for Beta distribution. Predictions of the neural network are treated as subjective opinions and learn the function that collects evidence by a deterministic neural network from data.

Domains 202 and 204 are marked as X and Y respectively in FIG. 2. Given sample i, let fₚₒₛ(X, A|θ) and f_{neg}(X, A|θ) represent the positive and negative evidence vectors predicted by EGNNs, where X is the input node features, A is an adjacency matrix 206, and θ represents network parameters. Then, the parameters of Beta distribution for node i and label k are:

αᵢₖ=fₚₒₛ(X, A|θ)+1,

βᵢₖ=f_{neg}(X, A|θ)+1,

With N training samples and K different classes, a multi-label evidential neural network is trained by minimizing Beta Loss 226:

$$\begin{matrix}{ℒ}_{Beta} = {\sum }_{i = 1}^{N} {\sum }_{k = 1}^{K} \int [ B C E ( {y}_{ik} , {p}_{i k} ) ] B ( {α}_{ik} , {β}_{i k} ) d {p}_{i k} \\ = {\sum }_{i = 1}^{N} {\sum }_{k = 1}^{K} \int [ - {y}_{i k} log ( {p}_{i k} ) - \\ ( 1 - {y}_{i k} ) log ( 1 - {ρ}_{i k} ) ] B ( {α}_{ik} , {β}_{i k} ) d {p}_{i k} \\ = {\sum }_{i = 1}^{N} {\sum }_{k = 1}^{K} [ - {y}_{i k} {E}_{pik \sim Beta} [ log ( {p}_{i k} ) ] - \\ ( 1 - {y}_{i k} ) {E}_{{p}_{ik} \sim Beta} [ log ( 1 - {p}_{i k} ) ] ]\end{matrix}$$

where B(αᵢₖ, βᵢₖ) is a 2-dimensional Beta function. BCE denotes the Binary Cross Entropy Loss. pᵢₖrepresents the predicted probability of sample i belonging to class k by model. yᵢₖ represents the ground truth for sample i with label k, e.g., yᵢₖ=1 means the training node i belongs to class k, otherwise yₖ=0.Eₚ_{_{ik˜Beta}}[log(pᵢₖ)] can be formulated and derived as follows:

$$\begin{matrix}{E}_{pik \sim Beta} [ log ( {p}_{i k} ) ] = {\int }_{0}^{1} log {p}_{i k} f ( {p}_{i k} ; {α}_{ik} , {β}_{i k} ) {dp}_{i k} \\ = {\int }_{0}^{1} log {p}_{i k} \frac{{{p}_{ik}^{{α}_{lk} - 1} ( 1 - {p}_{ik} )}^{{β}_{ik} - 1}}{B ( {α}_{lk} , {β}_{k k} )} {dp}_{ik} \\ = \frac{1}{B ( {α}_{ik} , {β}_{i k} )} {\int }_{0}^{1} \frac{\partial {{p}_{ik}^{{α}_{ik} - 1} ( 1 - {p}_{ik} )}^{{β}_{lk} - 1}}{\partial {α}_{i k}} d {p}_{i k} \\ = \frac{1}{B ( {α}_{ik} , {β}_{i k} )} \frac{\partial }{\partial {α}_{i k}} {\int }_{0}^{1} {{p}_{i k}^{α - 1} ( 1 - {p}_{i k} )}^{{β}_{i k - 1} - 1} {dp}_{i k} \\ = \frac{1}{B ( {α}_{ik} , {β}_{i k} )} \frac{\partial B ( {α}_{ik} , {β}_{i k} )}{\partial {α}_{i k}} \\ = \frac{\partial ln B ( {α}_{ik} , {β}_{i k} )}{\partial {α}_{ik}} \\ = \frac{\partial ln Γ ( {α}_{i k} )}{\partial {α}_{ik}} - \frac{\partial ln Γ ( {α}_{i k} + {β}_{i k} )}{\partial {α}_{ik}} \\ = ψ ( {α}_{i k} ) - ψ ( {α}_{i k} + {β}_{i k} )\end{matrix} ,$$

where Γ(⋅) represents the Gamma function. By the same derivation, we can obtain the term Eₚ_{ₖ}_{˜Beta}[log(1−pᵢₖ)]−Ω(βᵢₖ)−Ω(αᵢₖ+βᵢₖ). Thus, the Beta Loss 226 term [symbol] _{Beta} is:

$${ℒ}_{Beta} = {Σ}_{j = 1}^{N} {Σ}_{i = 1}^{K} [ {y}_{i j} ( ψ ( {α}_{i j} + {β}_{i j} ) - ψ ( {α}_{i j} ) ) + ( 1 - {y}_{i j} ) ( ψ ( {α}_{i j} + {β}_{i j} ) - ψ ( {β}_{i j} ) ) ] .$$

where Ω(⋅) denotes the Digamma function. Besides, as the belief and disbelief of label k for sample t, we have:

$${b}_{i k} = \frac{{α}_{ik} - 1}{{α}_{i k} + {β}_{i k}} ,$$

$${d}_{i k} = \frac{{β}_{i k} - 1}{{α}_{i k} + {β}_{i k}} .$$

For the following inference process, these beliefs 218 are regarded as multi-label opinions, to formulate a Joint Belief 220 and quantify OOD samples. So far, for in-distribution multi-label classification, we set the positive belief as the probability of class i for sample j, i.e.,

$$\frac{{α}_{ik} - 1}{{α}_{i k} + {β}_{i k}} ,$$

for time reauction.

After obtaining separate beliefs 218 of multiple labels, these beliefs 218 or opinions need to be combined and quantified in an integrated opinion, e.g., Opinions Fusion into Joint Belief 220. Note that, if a sample belongs to any label we already know, then it is an ID sample. In other words, only samples that do not belong to any known category should be classified as OOD samples. Hence, naive operations like summing up all the beliefs are inapplicable for multi-label settings.

Inspired by the multiplication in subjective logic, let X={x₁, x₂} and Y={y₁, y₂} be two different domains (202 and 204, respectively), and let ωₓ=(bₓ, dₓ, uₓ, aₓ) and ω_{y}=(b_{y}, d_{y},u_{y}, a_{y}) be binomial opinions on X and Y respectively. A is the adjacency matrix. Then, the joint opinion ω_{x∨y} is formulated as:

$${b}_{x ⋁ y} = {b}_{x} + {b}_{y} - {b}_{x} {b}_{y} .$$

$$\frac{{d}_{x ⋁ y} = {d}_{x} {d}_{y} + {a}_{x} ( 1 - {a}_{y} ) {d}_{x} {u}_{y} + ( 1 - {a}_{x} ) {a}_{y} {u}_{x} {d}_{y}}{{a}_{x} + {a}_{y} - {a}_{x} {a}_{y}} ,$$

$${u}_{x ⋁ y} = {u}_{x} {u}_{y} + \frac{{a}_{y} {d}_{x} {u}_{y} + {a}_{x} {u}_{x} {d}_{y}}{{a}_{x} + {a}_{y} - {a}_{x} {a}_{y}} ,$$

$${a}_{x ⋁ y} = {a}_{x} + {a}_{y} - {a}_{x} {a}_{y} .$$

Based on that, the Joint Belief 220 of a certain sample i is b_{1∨2∨ . . . ∨K}, which can be calculated recursively by b_{x∨y}=bₓ+b_{y}+b_{y}−bₓb_{y}. Only samples which do not belong to any known labels will have a relative low Joint Belief, which can effectively differentiate them from in-distribution samples. Thus, we use the Joint Belief to distinguish whether a sample 222 is in-distribution or a sample 223 is out-of-distribution.

With a higher Joint Belief, we are more confident to consider a sample as in-distribution sample. In useful embodiments, a Joint Belief Threshold can be set and employed to distinguish between in-distribution and out of distribution samples, nodes or graphs.

Kernel-based Node Positive Evidence Estimation (KNPE) 224 estimates prior Beta distribution parameters for each node based on the labels of training node and node-level distance. To be specific, the estimation of positive evidence \hat{α} is focused on.

For each pair of nodes i and j, calculate the node-level distance dᵢⱼ, i.e., the shortest path between nodes i and j. Then, the Gaussian kernel function is used to estimate the positive distribution effect between nodes i and j:

$$g ( {d}_{i j} ) = \frac{1}{σ \sqrt{2 π}} exp ( - \frac{{d}_{i j}^{2}}{2 {σ}^{2}} ) ,$$

where σ is the bandwidth parameter.

The contribution of positive evidence estimation for node j from training node i is hᵢⱼ(yᵢ, dᵢⱼ)=[hᵢⱼ¹, hᵢⱼ², . . . , hᵢⱼ^{k}, . . . , hᵢⱼ^{K}], where yᵢ=[yᵢ₁, . . . , yᵢₖ, . . . , y_{iK}]=[0,1]^{K} represents the in-distribution label vector of training node i. hᵢⱼ^{k} is obtained by:

$${h}_{i j}^{k} = \{ \begin{matrix}0 & {y}_{i k} = 0 \\ g ( {d}_{i j} ) & {y}_{i k} = 1\end{matrix} .$$

The prior positive evidence êⱼ is estimated as Σ_{i∈N}hᵢⱼ(yᵢ,dᵢⱼ), where N is the set of training samples and the prior positive parameter \hat{α}ⱼ=êⱼ+1. During the training process, the KL-divergence is minimized between model predictions of positive evidence (positive evidence loss ( [symbol] _{PE}) 230) [symbol] _{PE}=Σⱼ₌₁^{N} KL(\hat{α}ⱼ∥\hat{α}ⱼ). A total loss function 230 to optimize the model (minimization function (min)) includes:

min [symbol] ₜₒₜₐₗ= [symbol] _{Beta}+λ [symbol] _{PE} where λ denotes a trade-off parameter with [symbol] _{PE}.

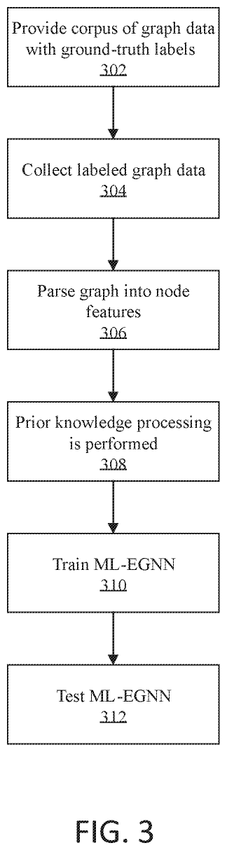

Referring to FIG. 3, a method for training a model for out-of-distribution node determination in graphs is illustratively shown and described. In block 302, a corpus of graph data with ground-truth labels of multi-labeled classes are provided as input. The goal is to detect the out-of-distribution nodes in the graphs using a Multi-Label Evidential Graph Neural Network (ML-EGNN) framework to address the node-level multi-label out-of-distribution problem. In block 304, labeled graph data is collected. Labeled graph data can include any type of information, e.g., social media network citation networks, drug interaction data, medical monitoring data. The labeled graph data can include a set of strongly labeled data with multi-class labels.

In block 306, a data processing device is employed to parse original graph data into its corresponding features. In one example, social media user information is collected as the node features. In another example, medical information is collected for individuals. In yet another example, data is collected for a Protein-Protein-Interaction (PPI) network.

In block 308, prior knowledge processing is performed by a computer processing device. A kernel density estimation method is employed to estimate pseudo labels for evidence labels. This process is employed to optimize the model based upon minimization of loss (e.g., beta and positive evidence loss).

In block 310, Multi-Label Evidential Graph Neural Networks training is performed. The ground-truth multi-labels are applied to train the ML-EGNNs for node-level multi-label out-of-distribution detection.

In block 312, multi-label out-of-distribution detection test is performed. A final predicted result is generated for both node classification and multi-label out-of-distribution based on the belief, disbelief and uncertainty outputs. A threshold can be set for classification criteria. This threshold will be dependent on confidence and the desired accuracy of the OOD classification.

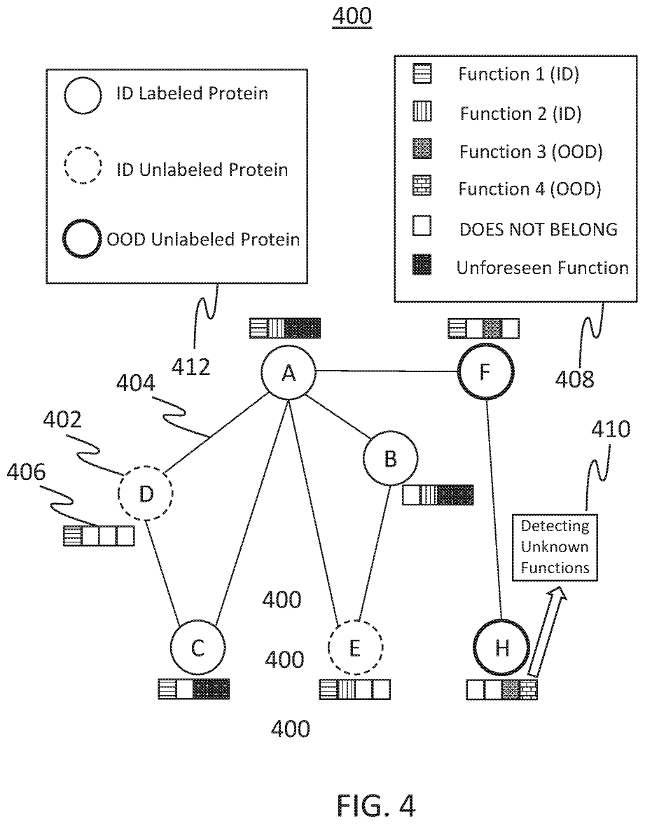

Referring to FIG. 4, an illustrative example of a Protein-Protein Interaction (PPI) network 400 is described employing a multi-label evidential graph neural network to improve the performance of node-level multi-label out-of-distribution detection. The PPI network 400 includes nodes 402 which are connected by edges 404. Each node includes labels 406 in a function block that in this example includes four functions or features. Each node in labeled with a letter A, B, C, D, E, F and H. The functions are identified using a key 408. The key 408 shows Function 1 and Function 2 as being In-Distribution (ID) functions and Function 3 and Function 4 as being Out-of-Distribution (OOD) functions. There are also a function category Does Not Belong and Unforeseen function.

A key 412 shows details about types of nodes. These include: ID Labeled Protein, ID Unlabeled Protein and OOD Unlabeled Protein. Function 3 and Function 4 are unseen for Labeled Nodes A, B and C. A traditional classification method will confidentially put OOD Unlabeled Nodes H and F into one or more In-Distribution Functions (like Function 1 and Function 2). This defect will lead to the model being unable to detect the unknown functions. Hence, it is necessary to study the OOD detection problem on a multi-label graph. In this way, the nodes having unknown functions or unforeseen or undiscovered label types can be discovered. Detecting multi-class OOD nodes on a graph is not the same as detecting OOD nodes in multi-label settings. For example, multi-class classification assigns each data sample one and only one label from more than two classes. Multi-label classification can be used to assign a number of labels to each data sample.

An uncertainty-based method may detect OOD proteins by higher uncertainty on Function 1 or Function 2. However, in this way, in-distribution node D may also have a high uncertainty score on Function 2 since it only has Function 1. Given that, those methods may misclassify some ID nodes into OOD samples when they have more sparse labels. Note that, we only consider OOD Unlabeled Nodes in which all the labels are unseen, e.g., nodes like F with both ID Labels and OOD Labels are out of consideration.

A novel multi-label opinion fusion enriched multi-label uncertainty representation with evidence information permits out-of-distribution prediction. Out-of-distribution detection with uncertainty estimation for graph settings with consideration of inherent multi-label properties of nodes and the ability to fuse information from different labels to distinguish OOD nodes enables the present embodiments to detect OOD nodes.

For the PPI network 400, nodes 402 represent proteins, edges 404 connect pairs of interacting proteins, labels 406 indicate different functions of proteins. There are three kinds of nodes: In-Distribution Labeled Proteins A, B and C for training; In-Distribution Unlabeled Proteins D and E; Out-of-Distribution Unlabeled Proteins F and H. During the training process, Functions 3 and 4 are unseen/unknown to the model. Node H is output as a detected OOD node as unknown functions 410 are detected. Upon detection, corrective actin can be taken, such as providing updates to label definitions, identifying the new or unknown functions, redefining or reclassifying the node, etc.

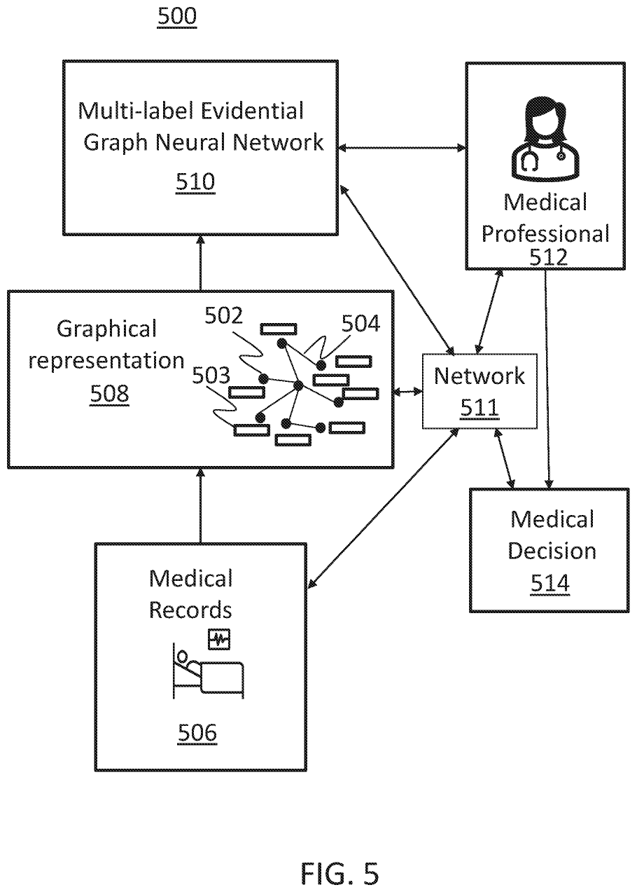

Referring to FIG. 5, an illustrative example of a medical system 500 that employs a multi-label evidential graph neural network to improve the performance of node-level multi-label out-of-distribution detection. The medical system 500 can include medical records 506 for multiple patients stored in memory on a server, in a cloud network, etc. The medical records 506 can be organized into a graphical representation 508. The graphical representation 508 can include nodes 502 connected by edges 504.

Each node 502 can represent a patient or user of the medical system 500, and the node feature can be considered as patient information, such as age, race, weight, etc. The edges 504 can represent relationships between users or relationships to other criteria, for example, the edges 504 can connect patients that share a doctor, a hospital or other commonality. For some nodes, the system includes associated labels, which have multiple classes (multi-class labels), such as specific medical diseases, e.g., diabetes, high blood pressure, heart stents, etc.

All this information constructs representative graphs as input for the ML-EGNN 510. The output of ML-EGNN 510 will be disease predictions for other patients who do not have labels. The prediction includes disease classifications and out-of-distribution detections (e.g., detection of new diseases). All of this information can be provided to medical professionals 512 over a network or medical computer system 511. The network can include an internal or external network (e.g., cloud). The medical professionals 512 can make medical decisions 514 based on this information. The medical professionals 512 can also use this information to update patient data and make the system models more accurate and efficient.

Each node 502 includes labels 503 associated with one or more features of each patient. In one example, labels 503 can include the features stored in the medical records 506, e.g., diagnoses for each patient, data collected for a particular medical condition, a medical history of each patient, etc. In one example, the labels 503 can include test data for tests accumulated over time, can include medical conditions, can include patient features or biological characteristics, etc. ML-EGNN 510 that has been trained to predict out-of-distribution nodes is employed to predict test results, medical conditions, doctor reports or other information that is likely Out-of-Distribution (OOD).

Multi-label opinion fusion enriched multi-label uncertainty representation with evidence information permits out-of-distribution prediction by the Multi-Label Evidential Graph Neural Network 510. Out-of-distribution detection with uncertainty estimation for graph settings, provides the ability to distinguish and detect OOD nodes. In this way, OOD nodes or features including unforeseen or rare medical information can be identified for further analysis and consideration by healthcare workers and/or medical professionals 512. By identifying OOD features including unforeseen or rare medical information, misclassification of patient records, patient medical history, etc. can be prevented. The discovered OOD features can be properly labeled for future consideration and the features which could have otherwise been misclassified can be considered and employed in improving medical decisions 514 by medical professionals 512.

The network 511 can interact with any piece of the system and convey information and resources as needed to identify OOD nodes, update OOD nodes, display updates of patient information, record medical professional inputs/decisions, etc. Information can be conveyed over the network 511 so that the information is available to all users. The functionality provided for determining OOD nodes can be provided as a service for medical staff and programmers to update patient's profiles in a distributed network setting, in a hospital setting, in a medical office setting, etc.

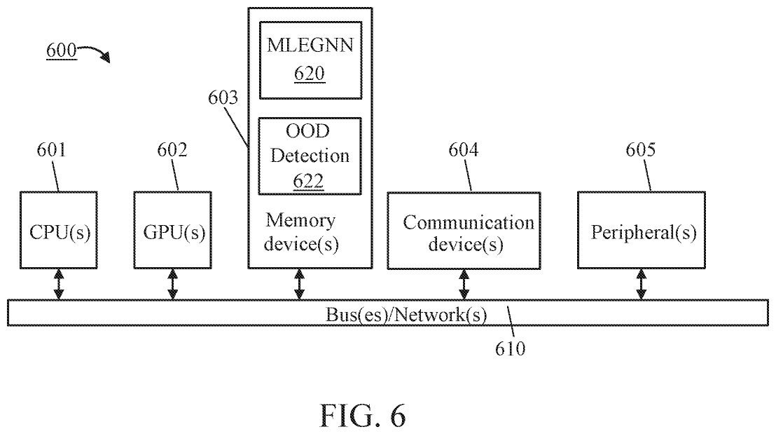

Referring to FIG. 6, a block diagram showing an exemplary processing system 600 employed in accordance with an embodiment of the present invention. The processing system 600 can include one or more computer processing units (e.g., CPUs) 601, one or more graphical processing units (GPUs) 602, one or more memory devices 603, communication devices 604, and peripherals 605. The CPUs 601 can be single or multi-core CPUs. The GPUs 602 can be single or multi-core GPUs. The CPUs and/or GPUs can be, in whole or part, hardware processing subsystems. The one or more memory devices 603 can include caches, RAMs, ROMs, and other memories (flash, optical, magnetic, etc.). The communication devices 604 can include wireless and/or wired communication devices (e.g., network (e.g., WIFI, etc.) adapters, etc.). The peripherals 605 can include a display device, a user input device, a printer, an imaging device, and so forth. Elements of processing system 600 are connected by one or more buses or networks (collectively denoted by reference numeral 610).

In an embodiment, memory devices 603 can store specially programmed software modules to transform the computer processing system into a special purpose computer configured to implement various aspects of the present invention. In an embodiment, special purpose hardware (e.g., Application Specific Integrated Circuits, Field Programmable Gate Arrays (FPGAs), and so forth) can be used to implement various aspects of the present invention.

In an embodiment, memory devices 603 store program code for implementing node level out-of-distribution detection on multi-label graph data. A ML-EGNN 620 can be stored in memory 603 along with program code for OOD detection 622 to enable efficient multi-label node classification and out-of-distribution detection of nodes in a graphical network.

The processing system 600 may also include other elements (not shown), for example, various other input devices and/or output devices can be included in processing system 600, depending upon the particular implementation. Wireless and/or wired input and/or output devices can be employed. Moreover, additional processors, controllers, memories, and so forth, in various configurations can also be utilized. These and other variations of the processing system 600 can also be provided.

Moreover, it is to be appreciated that various figures as described below with respect to various elements and steps relating to the present invention that may be implemented, in whole or in part, by one or more of the elements of system 600.

A MLEGNN is an information processing system that is inspired by biological nervous systems, such as the brain. MLEGNNs includes an information processing structure, which includes a large number of highly interconnected processing elements (called “neurons” or “nodes”) working in parallel to solve specific problems. MLEGNNs are furthermore trained using a set of training data, with learning that involves adjustments to weights that exist between the neurons. Here, the MLEGNNs is configured for a specific application, such as classification of nodes by fusing opinions to arrive at a Joint Belief, through such a learning process.

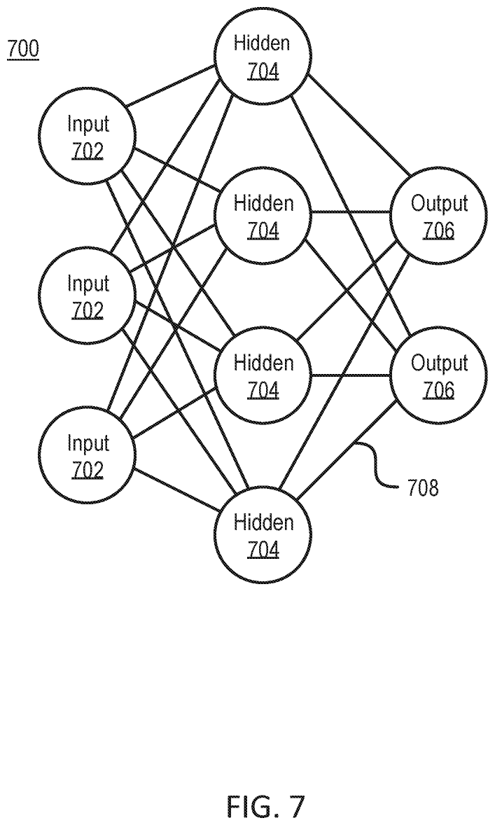

Referring now to FIG. 7, an illustrative diagram of a neural network 700 is shown. Although a specific structure is shown, having three layers and a set number of fully connected neurons, it should be understood that this is intended solely for the purpose of illustration. In practice, the present embodiments may take any appropriate form, including any number of layers and any pattern or patterns of connections therebetween.

MLEGNNs demonstrate an ability to derive meaning from complicated or imprecise data and can be used to extract patterns and detect trends that are too complex to be detected by humans or other computer-based systems. The structure of a neural network is known generally to have input neurons 702 that provide information to one or more “hidden” neurons 704. Connections 708 between the input neurons 702 and hidden neurons 704 are weighted, and these weighted inputs are then processed by the hidden neurons 704 according to some function in the hidden neurons 704. There can be any number of layers of hidden neurons 704, and as well as neurons that perform different functions. There exist different neural network structures as well, such as a convolutional neural network, a maxout network, etc., which may vary according to the structure and function of the hidden layers, as well as the pattern of weights between the layers. The individual layers may perform particular functions, and may include convolutional layers, pooling layers, fully connected layers, softmax layers, or any other appropriate type of neural network layer. With respect to MLEGNNs in accordance with present embodiments, the layers of the MLEGNN include graph convolutional layers, fully connected layers, a ReLU layer. A set of output neurons 706 accepts and processes weighted input from the last set of hidden neurons 704.

This represents a “feed-forward” computation, where information propagates from input neurons 702 to the output neurons 706. Upon completion of a feed-forward computation, the output is compared to a desired output available from training data. The error relative to the training data is then processed in “backpropagation” computation, where the hidden neurons 704 and input neurons 702 receive information regarding the error propagating backward from the output neurons 706. Once the backward error propagation has been completed, weight updates are performed, with the weighted connections 708 being updated to account for the received error. It should be noted that the three modes of operation, feed forward, back propagation, and weight update, do not overlap with one another. This represents just one variety of computation, and that any appropriate form of computation may be used instead.

To train an MLEGNNs, training data can be divided into a training set and a testing set. The training data includes pairs of an input and a known output. During training, the inputs of the training set are fed into the MLEGNNs using feed-forward propagation. After each input, the output of the MLEGNNs is compared to the respective known output. Discrepancies between the output and the known output that is associated with that particular input are used to generate an error value, which may be backpropagated through the MLEGNNs, after which the weight values of the MLEGNNs may be updated. This process continues until the pairs in the training set are exhausted.

After the training has been completed, the MLEGNNs may be tested against the testing set, to ensure that the training has not resulted in overfitting. If the ML-EGNNs can generalize to new inputs, beyond those which it was already trained on, then it is ready for use. If the MLEGNNs does not accurately reproduce the known outputs of the testing set, then additional training data may be needed, or hyperparameters of the MLEGNNs may need to be adjusted.

MLEGNNs may be implemented in software, hardware, or a combination of the two. For example, each weight 708 may be characterized as a weight value that is stored in a computer memory, and the activation function of each neuron may be implemented by a computer processor. The weight value may store any appropriate data value, such as a real number, a binary value, or a value selected from a fixed number of possibilities, that is multiplied against the relevant neuron outputs.

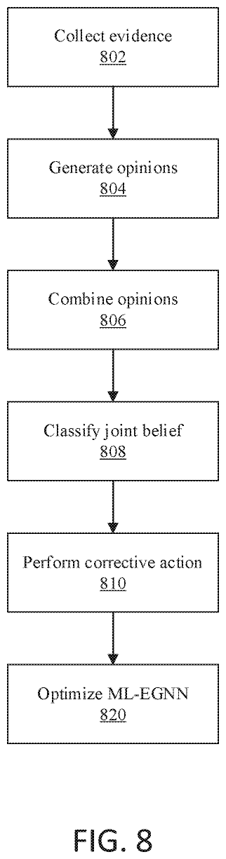

FIG. 8 is a flow diagram illustrating a method for detecting out-of-distribution nodes in graphs, in accordance with an embodiment of the present invention. The method preferable employs evidential deep learning to provide better predictions/discovery for OOD nodes. Once discovered, OOD nodes can be pruned from a graph, updated with labels, reclassified or subjected to other corrective actionts). Removing, reclassifying, labeling such OOD nodes not only improves the data set but also improves computer processing time when using the graph for practical applications such as, medical decisions, drug interactions, etc.

In block 802, evidence is collected to quantify predictive uncertainty of diverse labels of nodes in a graph of nodes and edges using positive evidence from labels of training nodes of a multi-label evidential graph neural network. The collection of evidence to quantify predictive uncertainty can include predicting positive and negative evidence vectors from the multi-label evidential graph neural network.

The positive and negative evidence vectors can be employed during training to generate a beta distribution using the positive and negative evidence vectors wherein the beta distribution is used to train the multi-label evidential graph neural network by minimizing beta loss.

In block 804, multi-label opinions including belief and disbelief are generated for the diverse labels. The multi-label opinions can include computing for sample i, class k:

$${b}_{k} = \frac{{α}_{k} - 1}{{α}_{k} + {β}_{k}} and {d}_{k} = \frac{{β}_{k} - 1}{{α}_{k} + {β}_{k}} ,$$

where bₖ indicates positive belief mass distribution, dₖ indicates negative belief mass distribution, αₖ and βₖ are features of positive and negative evidence vectors, respectively.

In block 806, the opinions are combined into a joint belief by employing a comultiplication operation of binomial opinions. The combination of opinions into a joint belief can include combining belief opinions b of a sample by b_{1∨2∨ . . . ∨K} calculated recursively by b_{x∨y}=bₓ+b_{y}−bₓb_{y}.

In block 808, the joint belief is classified to detect out-of-distribution nodes of the graph, wherein classifying the joint belief to detect out-of-distribution nodes of the graph can include determining whether the joint belief exceeds a threshold value for a given node to determine if the node is out-of-distribution.

In block 810, a corrective action responsive to a detection of an out-of-distribution node is performed. The corrective actin can include automatically assigning or applying a new label to the OOD node. In another embodiment, the node can be classified in a new class. In other embodiments, e.g., where the nodes include patient information, the corrective action can include alerting medical personnel of the out-of-distribution node. A medical decision may be needed based on the out-of-distribution node. For example, if given test results are unknown or unlabeled for a particular patient, a system in accordance with the present embodiment could identify the OOD node and send an alert to a healthcare worker. A decision on whether to take action, e.g., recommend a test, prescribe a drug, isolate the patient can accordingly be made.

In block 820, a neural network can be initially or continuously trained by optimizing the multi-label evidential graph neural network by minimizing total loss which includes a beta loss component and a positive evidence loss component. This can be achieved through a kernel-based evidence estimation process.

As employed herein, the term “hardware processor subsystem” or “hardware processor” can refer to a processor, memory, software or combinations thereof that cooperate to perform one or more specific tasks. In useful embodiments, the hardware processor subsystem can include one or more data processing elements (e.g., logic circuits, processing circuits, instruction execution devices, etc.). The one or more data processing elements can be included in a central processing unit, a graphics processing unit, and/or a separate processor- or computing element-based controller (e.g., logic gates, etc.). The hardware processor subsystem can include one or more on-board memories (e.g., caches, dedicated memory arrays, read only memory, etc.). In some embodiments, the hardware processor subsystem can include one or more memories that can be on or off board or that can be dedicated for use by the hardware processor subsystem (e.g., ROM, RAM, basic input/output system (BIOS), etc.).

In some embodiments, the hardware processor subsystem can include and execute one or more software elements. The one or more software elements can include an operating system and/or one or more applications and/or specific code to achieve a specified result.

In other embodiments, the hardware processor subsystem can include dedicated, specialized circuitry that performs one or more electronic processing functions to achieve a specified result. Such circuitry can include one or more application-specific integrated circuits (ASICs), field-programmable gate arrays (FPGAs), and/or programmable logic arrays (PLAs). These and other variations of a hardware processor subsystem are also contemplated in accordance with embodiments of the present invention.

Reference in the specification to “one embodiment” or “an embodiment” of the present invention, as well as other variations thereof, means that a particular feature, structure, characteristic, and so forth described in connection with the embodiment is included in at least one embodiment of the present invention. Thus, the appearances of the phrase “in one embodiment” or “in an embodiment”, as well any other variations, appearing in various places throughout the specification are not necessarily all referring to the same embodiment. However, it is to be appreciated that features of one or more embodiments can be combined given the teachings of the present invention provided herein.

It is to be appreciated that the use of any of the following “/”, “and/or”, and “at least one of”, for example, in the cases of “A/B”, “A and/or B” and “at least one of A and B”, is intended to encompass the selection of the first listed option (A) only, or the selection of the second listed option (B) only, or the selection of both options (A and B). As a further example, in the cases of “A, B, and/or C” and “at least one of A, B, and C”, such phrasing is intended to encompass the selection of the first listed option (A) only, or the selection of the second listed option (B) only, or the selection of the third listed option (C) only, or the selection of the first and the second listed options (A and B) only, or the selection of the first and third listed options (A and C) only, or the selection of the second and third listed options (B and C) only, or the selection of all three options (A and B and C). This may be extended for as many items listed.

The foregoing is to be understood as being in every respect illustrative and exemplary, but not restrictive, and the scope of the invention disclosed herein is not to be determined from the Detailed Description, but rather from the claims as interpreted according to the full breadth permitted by the patent laws. It is to be understood that the embodiments shown and described herein are only illustrative of the present invention and that those skilled in the art may implement various modifications without departing from the scope and spirit of the invention. Those skilled in the art could implement various other feature combinations without departing from the scope and spirit of the invention. Having thus described aspects of the invention, with the details and particularity required by the patent laws, what is claimed and desired protected by Letters Patent is set forth in the appended claims.

What is claimed is:

1. A computer-implemented method for out-of-distribution detection of nodes in a graph, comprising:
collecting evidence to quantify predictive uncertainty of diverse labels of nodes in a graph of nodes and edges using positive evidence from labels of training nodes of a multi-label evidential graph neural network;
generating multi-label opinions including belief and disbelief for the diverse labels;
combining the opinions into a joint belief by employing a comultiplication operation of binomial opinions;
classifying the joint belief to detect out-of-distribution nodes of the graph; and
performing a corrective action responsive to a detection of an out-of-distribution node.

2. The method as recited in claim 1, wherein collecting evidence to quantify predictive uncertainty includes predicting positive and negative evidence vectors from the multi-label evidential graph neural network.

3. The method as recited in claim 2, wherein predicting the positive and negative evidence vectors includes generating a beta distribution using the positive and negative evidence vectors wherein the beta distribution is used to train the multi-label evidential graph neural network by minimizing beta loss in accordance with evidential deep learning.

4. The method as recited in claim 1, wherein generating multi-label opinions includes computing for sample i, class k:
$${b}_{k} = \frac{{α}_{k} - 1}{{α}_{k} + {β}_{k}} and {d}_{k} = \frac{{β}_{k} - 1}{{α}_{k} + {β}_{k}} ,$$
where bₖ indicates positive belief mass distribution, dₖ indicates negative belief mass distribution, αₖ and βₖ are features of positive and negative evidence vectors, respectively.

5. The method as recited in claim 1, wherein combining the opinions into a joint belief includes combining belief opinions b of a sample by b_{1∨2∨ . . . ∨K} calculated recursively by b_{x∨y}=bₓ+b_{y}−bₓb_{y}.

6. The method as recited in claim 1, wherein classifying the joint belief to detect out-of-distribution nodes of the graph includes determining whether the joint belief exceeds a threshold value for a given node to determine if the node is out-of-distribution.

7. The method as recited in claim 1, wherein the nodes include patient information, the corrective action includes:
alerting medical personnel of the out-of-distribution node; and
making a medical decision based on the out-of-distribution node.

8. The method as recited in claim 1, further comprising optimizing through training the multi-label evidential graph neural network by minimizing total loss which includes a beta loss component and a positive evidence loss component.

9. The method as recited in claim 1, wherein the corrective action includes:
applying a label to the out-of-distribution node.

10. The method as recited in claim 1, wherein the multi-label evidential graph neural network applies evidential deep learning.

11. A system for out-of-distribution detection of nodes in a graph, comprising:
a hardware processor; and
a memory that stores a computer program which, when executed by the hardware processor, causes the hardware processor to:
collect evidence to quantify predictive uncertainty of diverse labels of nodes in a graph of nodes and edges using positive evidence from labels of training nodes of a multi-label evidential graph neural network;
generate multi-label opinions including belief and disbelief for the diverse labels;
combine the opinions into a joint belief by employing a comultiplication operation of binomial opinions;
classify the joint belief to detect out-of-distribution nodes of the graph; and
perform a corrective action responsive to a detection of an out-of-distribution node.

12. The system as recited in claim 11, wherein the computer program further causes the hardware processor to collect evidence to quantify predictive uncertainty by predicting positive and negative evidence vectors from the multi-label evidential graph neural network.

13. The system as recited in claim 12, wherein the computer program further causes the hardware processor to generate a beta distribution using the positive and negative evidence vectors wherein the beta distribution is used to train the multi-label evidential graph neural network by minimizing beta loss in accordance with evidential deep learning.

14. The system as recited in claim 11, wherein the computer program further causes the hardware processor to generate multi-label opinions by computing for sample i, class k:
$${b}_{k} = \frac{{α}_{k} - 1}{{α}_{k} + {β}_{k}} and {d}_{k} = \frac{{β}_{k} - 1}{{α}_{k} + {β}_{k}} ,$$
where bₖ indicates positive belief mass distribution, dₖ indicates negative belief mass distribution, αₖ and βₖ are features of positive and negative evidence vectors, respectively.

15. The system as recited in claim 11, wherein the computer program further causes the hardware processor to combine the opinions into a joint belief by combining belief opinions b of a sample by b_{1∨2∨ . . . ∨K} calculated recursively by b_{x∨y}=bₓ+b_{y}−bₓb_{y}, wherein the computer program further causes the hardware processor to classify the joint belief to detect out-of-distribution nodes of the graph by determining whether the joint belief exceeds a threshold value for a given node to determine if the node is out-of-distribution.

16. The system as recited in claim 11, wherein the nodes include patient information and the computer program further causes the hardware processor to:
alert medical personnel of the out-of-distribution node to enable a medical decision based on the out-of-distribution node.

17. The system as recited in claim 11, wherein the computer program further causes the hardware processor to optimize the multi-label evidential graph neural network through training by minimizing total loss which includes a beta loss component and a positive evidence loss component.

18. The system as recited in claim 11, wherein the corrective action includes applying a label to the out-of-distribution node.

19. A computer program product for out-of-distribution detection of nodes in a graph, the computer program product comprising a non-transitory computer readable storage medium having program instructions embodied therewith, the program instructions executable by a computer to cause the computer to perform a method comprising:
collecting evidence to quantify predictive uncertainty of diverse labels of nodes in a graph of nodes and edges using positive evidence from labels of training nodes of a multi-label evidential graph neural network;
generating multi-label opinions including belief and disbelief for the diverse labels;
combining the opinions into a joint belief by employing a comultiplication operation of binomial opinions;
classifying the joint belief to detect out-of-distribution nodes of the graph; and
performing a corrective action responsive to a detection of an out-of-distribution node.

20. The computer program product as recited in claim 19, wherein the nodes include patient information and the corrective action includes:
alert medical personnel of the out-of-distribution node to enable a medical decision based on the out-of-distribution node.
